(19) United States
(12) Patent Application Publication
(10) Pub. No.: US 2012/0122716 A1
(43) Pub. Date: May 17, 2012

(54) CELL LINE DERIVED FROM THE EPITHELIAL LINING OF THE HUMAN ENDOLYMPHATIC SAC IN THE INNER EAR

(75) Inventors: DAVID LIM, PASADENA, CA (US); SUNG-KYUN MOON, FULLERTON, CA (US); HELGE RASK-ANDERSEN, Uppsala (SE); JOHNG S. RHIM, POTOMAC, MD (US)

(73) Assignee: DAVID LIM, PASADENA, CA (US)

(21) Appl. No.: 13/296,136

(22) Filed: Nov. 14, 2011

Related U.S. Application Data
(60) Provisional application No. 61/413,288, filed on Nov. 12, 2010.

(51) Int. Cl. C12Q 1/68; C12N 15/86; C12N 15/867; G01N 27/62; C12N 5/10; C12Q 1/02; C40B 30/04; C12N 15/85; C12N 15/861

(52) U.S. Cl. 506/9; 435/467; 435/371; 435/6.12; 435/29; 435/6.1; 324/459

(57) ABSTRACT

In some embodiments, the invention relates to a stable, long-term human ES cell line. In other aspects, the invention relates to methods for establishing a stable long-term ES cell line and methods for screening therapeutic treatments for inner ear diseases, such as Meniere's disease.

### RELATED APPLICATIONS

This application claims the benefit of U.S. Provisional Application No. 61/413,288 filed Nov. 12, 2010, which is hereby incorporated by reference in its entirety.

### BACKGROUND OF THE INVENTION

1. Field of the Invention

In one aspect, this invention relates to the establishment and characterization of a human endolymphatic sac epithelial cell line. In another aspect this invention relates to methods of screening for biomarkers for diseases related to the inner ear.

2. Description of the Related Art

The endolymphatic sac (ES), which is a part of the inner ear, is believed to absorb endolymph, the fluid contained in the membranous labyrinth of the inner ear. Surgical blockage of the ES and the endolymphatic duct causes accumulation of endolymph in the cochlea and vestibule, as so-called endolymphatic hydrops (the net accumulation of water in the inner ear endolymphatic space). Meniere's disease is nearly invariably associated with endolymphatic hydrops. Endolymphatic hydrops in the cochlea causes deafness, while endolymphatic hydrops in the vestibule causes vertigo (Sakikawa, Y, et al. 1999 Ann Owl Rhinol Laryngol 108: 271-276; and Tonndorf, J. 1976 Arch Otorhinolaryngol 212: 293-299). Endolymph regulation in the inner ear is thus important for hearing and the sense of equilibrium.

Primary culture of the human endolymphatic sac (ES) epithelial cells has previously been reported (Kim et al. 2009 Otology & Neurotology 30:529-534; and Linder B et al., 2001 Otology & Neurotology 22: 938-43). However, a stable cell line of human ES cells has not been reported.

### SUMMARY OF THE INVENTION

Human ES cells are an essential tool for investigation of inner ear diseases. In the past, researchers have had to rely on primary cultures of these cells for research. Such cultures have been of limited value, however, for the following reasons: 1) human ES cells are difficult to obtain, 2) very small numbers of cells can be obtained and cultured, 3) the cultures can be maintained for short periods of time and die quickly, and 4) fibroblasts often overgrow the culture.

The establishment of a human ES cell line is therefore of significant value as it obviates the need for using primary cultures and enables scientists to perform studies that would not have been possible using primary cultures. The human ES cell line disclosed herein can be used for studies, including those aimed at understanding inner ear fluid regulation, ion transport, secretory function and immune functions.

We have established and characterized, for the first time, a stable long-term human ES cell line. The establishment of such a cell line provides a novel method to study diseases of the inner ear, such as Meniere's disease, and is envisioned to serve as a useful tool to test therapeutic approaches in disease models.

Therefore, in one aspect, the invention relates to a stable, long-term human ES cell line. In one preferred embodiment, the invention relates to the human ES cell line on deposit as ATCC Accession # ______.

In another aspect, the invention relates to a method for establishing a stable long-term ES cell line. The method comprises: isolating ES cells from a ES tissue by enzymatic digestion; plating the ES cells in laminin-coated tissue culture dishes in a medium comprising insulin, progesterone, and heregulin; immortalizing the ES cells by exposure to a retrovirus construct comprising the human papilloma virus E6-E7 genes and the Neo^{r} gene; and selecting immortal cells by resistance to neomycin. In some embodiments, the medium may further comprise at least one additional component selected from the group consisting of bovine pituitary extract, transferrin, α-tocopherol, and forskolin.

In yet another aspect, the invention relates to a method for screening therapeutic treatments for inner ear diseases, such as Meniere' s disease. The screening method comprises: exposing ES cells of a human ES cell line of the present invention to a therapeutic treatment; and monitoring an index of ES cell function. In preferred embodiments of the screening method, the index of ES cell function is monitored by an analysis selected from the group consisting of morphological changes, growth rate, immunohistochemical staining and ionic transport.

### BRIEF DESCRIPTION OF THE DRAWINGS

FIG. 1: Schematic diagram of mastoid-E-tube-middle ear complex. Mastoid cavity is continuous to the middle ear cavity, which is connected to eustachian tube which is consisted of bony part and cartilaginous part, where mixed glands are well developed. The cartilaginous E-tube, which is open to the pharynx, is closed by tubal cartilage and opens intermittently to aerate and equalize pressure between middle ear cavity and ambient pressure of the oropharynx. Poor tubal function is one of the major risk factors for otitis media susceptibility. The lining of the middle ear epithelium is covered by ciliated cells and secretory cells typical of respiratory epithelium. The mastoid cavity is largely covered by simple squamous mucosal epithelium. The eustachian tube is covered by tall columnar epithelial cells consists of ciliated and secretory cells.

FIG. 2: Middle ear cavity is covered with respiratory epithelium consisting of ciliated cells (arrowheads), secretory cells, and nonsecretory cells. Viral upper respiratory infection destroys these respiratory epithelial cells impairing mucociliary transport system essential for mucosal protection

FIG. 3: A SEM photomicrograph of surface view of chinchilla organ of Corti showing complexity of cell structures including one row of inner hair cells (IHC) and three rows of outer hair cells (OH1-OH3), inner phalangeal cells (IPx), outer phalangeal cells (OPx), and three rows of Deiter cells (D1-D3). The sensory cell hair bundles (stereocilia), arranged in “W” formation, are exposed to the endolymph (scala media).

FIG. 4: Epithelial lining of the endolymphatic sac consists of mitochondria-rich light cell (M) and ribosome-rich dark cell (R). There are also considerable variations of luminal cell surface areas among different cells and extent of intercellular space (ICS) indicating various stages of fluid transport. This may represent dynamic states of the fluid transport (absorption) at any given time. Loose subepithelial connective tissue contains capillaries (arrows) and nerve fibers (not shown), and has extensive network of fibrocyte processes (F) contacting to each other. Insert is a diagram of the endolymphatic sac and a line indicates the plane of section.

FIG. 5: SEM photomicrograph of ES surface showing two types of epithelial cells: cell with extensive microvilli (M) and cell with smooth surface (S), and free-floating macrophages (Ma) in the luminal surface. The cell with extensive microvilli is interpreted to be mitochondria-rich light cell and the cell with smooth surface is ribosome-rich dark cell.

FIG. 6: Schematic illustration summarizes signaling pathways involved in the individual and synergistic effects of IL-1 alpha and nontypeable H. influenzae (NTHi) on beta defensin 2 (DEFB4) transcription in the middle ear epithelial cells.

### DETAILED DESCRIPTION OF THE PREFERRED EMBODIMENT

The invention relates to an immortalized, human endolymphatic sac (ES) cell line, comprising two or more types of mucosal endothelial-like cells, containing an exogenous immortalizing gene. Methods of preparing the cell line and methods of using the cell line are included in the invention.

The (ES) cell line disclosed herein provides an in vitro model of human ES epithelial cells, enabling elucidation of a role of ES in inner ear fluid and ionic regulation. The cell line also permits investigation of cellular and molecular pathogenesis of inner ear diseases such as Meniere's disease.

### Definitions

The transitional term “comprising” is synonymous with “including,” “containing,” or “characterized by,” is inclusive or open-ended and does not exclude additional, unrecited elements or method steps.

The transitional phrase “consisting of” excludes any element, step, or ingredient not specified in the claim, but does not exclude additional components or steps that are unrelated to the invention such as impurities ordinarily associated therewith.

The transitional phrase “consisting essentially of” limits the scope of a claim to the specified materials or steps and those that do not materially affect the basic and novel characteristic(s) of the claimed invention.

The term “cell line,” as used herein, refers to individual cells, harvested cells, and cultures containing the cells, so long as they are derived from cells of the cell line referred to. A cell line is said to be “continuous,” “immortal,” or “stable” if the line remains viable over a prolonged time, typically at least about six months. To be considered a cell line, as used herein, the cells must remain viable for at least 40 passages.

As used in this application, the term “vector” refers to DNA or RNA vehicle, such as a plasmid, comprising nucleotide sequences enabling replication of the DNA or RNA in a suitable host cell. In this invention, a vector preferably includes a recombinant retrovirus containing oncogenes which are transcribed into mRNA and translated into proteins when the proviral sequence is expressed in the genetically modified target cell.

“Transfection” refers to the introduction of an exogenous nucleotide sequence, such as DNA vectors in the case of mammalian target cells, into a target cell whether or not any coding sequences are ultimately expressed. Numerous methods of transfection are known to those skilled in the art, such as: chemical methods (e.g., calcium-phosphate transfection), physical methods (e.g., electroporation, microinjection, particle bombardment), fusion (e.g., liposomes), receptor-mediated endocytosis (e.g., DNA-protein complexes, viral envelope/capsid-DNA complexes) and by biological infection by viruses such as recombinant viruses (Wolff, J. A. ed, Gene Therapeutics, Birkhauser, Boston, USA 1994). In the case of infection by retroviruses, the infecting retrovirus particles are absorbed by the target cells, resulting in reverse transcription of the retroviral RNA genome and integration of the resulting provirus into the cellular DNA. Genetic modification of the target cell is the indicia of successful transfection. “Genetically modified cells” refers to cells whose genotypes have changed as a result of cellular uptakes of exogenous nucleotide sequence by transfection. It will be appreciated that, as used herein, reference to “transfected cells” or “genetically modified cells” includes both the particular cell into which a vector or polynucleotide is introduced and progeny of that cell. “Primary cells” are cells that have been harvested from the tissue of an organism.

Some embodiments of the invention relate to a method for producing an immortalized human ES cell line, which comprises providing primary human ES cells in a culture and transfecting the human ES cells in the culture with an exogenous immortalizing gene so that the cell line is immortalized. In one embodiment, the exogenous immortalizing gene is a human papilloma virus gene.

The primary ES cells to be immortalized by the method of the present invention can be from one or more donors and/or cell sources.

Human ES cells are immortalized using the entire Human Papillomavirus (HPV) genome or portions thereof. The HPV DNA may be obtained from different strains of HPV which are associated with cancer. The HPV DNA may be obtained from different strains of HPV which are isolated from malignant or benign tumors taken from different tissues of humans. Examples of such strains of HPV include but are not limited to HPV-16, 18, 31, 33 and 35.

In one embodiment the cells are immortalized using the entire HPV genome from HPV-16, or the like. In another embodiment, the human ES cells are immortalized using at least the E7 DNA portion of the genome or at least the E6 DNA portion of the genome or combinations thereof. In one embodiment a DNA sequence homologous or significantly homologous to the DNA sequence of E6 or E7 of HPV is used to immortalize human ES cells. In another embodiment, the cells are immortalized using at least the E7 DNA portion in combination with the E6 DNA portion of the HPV-16 genome.

A preferred cell line was deposited under the Budapest Treaty on ______ with the American Type Culture Collection (10801 University Blvd., Manassas, Va.) as ATCC # ______.

### Deposit of Biological Material

A cell line termed was deposited as ATCC Accession No. on with the American Type Culture Collection (ATCC), 10801 University Blvd., Manassas, Va. 20110-2209, USA. This deposit was made under the provisions of the Budapest Treaty on the International Recognition of the Deposit of Microorganisms for the Purposes of Patent Procedure and the Regulations there under (Budapest Treaty). This assures maintenance of a viable culture of the deposit for 30 years from date of deposit. The deposit will be made available by ATCC under the terms of the Budapest Treaty, and subject to an agreement between Applicant and ATCC which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 USC §122 and the Commissioner's rules pursuant thereto (including 37 CFR §1.14). Availability of the deposited biological material is not to be construed as a license to practice the invention in contravention of the rights granted under the authority of any government in accordance with its patent laws.

Immortalized cell lines

The stable, immortalized cell lines of the invention are derived from human ES cells. Immortalized cells are preferred over primary cells for use as a testing system because of greater reproducibility of results and less onerous preparation for use (once an immortalized cell line has been established). Immortalized cell lines, derived from human endolymphatic sac tissues serve as model systems for the respective tissues from which they were derived. Immortalized cells are particularly advantageous because of their similarity to normal tissue cells.

An immortalized cell line is prepared from cells obtained from a specific tissue of a single human donor. A homolog of a given cell line is a second cell line prepared by the same method from the same tissue, but from a different donor. Different clonal isolates of a cell line are referred to as derivative cell lines.

### Expression Systems

Immortalizing genes can be transferred into the cell lines by transfection of plasmid DNA or by retroviral infection. A viral vector is preferably replication defective so that stable cell lines expressing immortalizing genes are obtained. Transfection of cells can occur through methods commonly used, such as calcium or strontium phosphate treatment, microinjection, electroporation, or lipofection. For example, the cells may be infected with a molony-LTR driven promoter or a vaccinia virus or lipofected with an adenovirus-promoter, HIV-promoter or CMV-promoter construct. The transfected DNA plasmid can contain a selectable marker gene or be co-transfected with a plasmid containing a selectable marker, and in some cases, the retroviral vector contains a selectable marker gene. Where one or more selectable markers are transferred into the cells along with the immortalizing gene, the cell populations containing the immortalizing gene can be identified and enriched by selecting for the marker or markers. Markers typically are antibiotic resistant to such antibiotics as tetracycline, hygromycin, neomycin, and the like.

In a preferred embodiment, the vector contains a bacterial neomycin resistance gene (Neo^{r}) as such a marker. Cells carrying Neo^{r} may be selected by means known to those of ordinary skill in the art, such as the addition of 100-200 μg/mL G418 to the growth medium. It will be readily apparent that other markers may be employed, and appropriate selections may be readily performed by those of ordinary skill in the art.

Retroviral vectors are the preferred vectors of this invention, though other viral vectors may be used, such as adenoviral vectors. Though adenoviral vectors have the advantage of not requiring dividing cells for transfection, they have a disadvantage in that they do not integrate into the genome, possibly making it more difficult to derive stable cell lines. Adeno-associated viral (AAV) vectors might also be used but have the disadvantage of a smaller packaging limit than retroviral vectors.

The retroviral vector can be any that is known in the art. Retroviruses to be adapted for use in accordance with this invention can be derived from many avian or mammalian hosts. However, a requirement for use is that the virus be capable of infecting cells, which are to be the recipients of the new genetic material (oncogene and/or desired gene) to be transduced using the retroviral vectors. Examples of retroviruses include avian retroviruses, such as avian erythroblastosis virus (AMV), avian leukosis virus (ALV), avian myeloblastosis virus (ABV), avian sarcoma virus (ACV), Fujinami sarcoma virus (FuSV), spleen necrosis virus (SNV), and Rous sarcoma virus (RSV). Non-avian viruses include: bovine leukemia virus (BLV); feline retroviruses such as feline leukemia virus (FeLV) or feline sarcoma virus (FeSV); murine retroviruses such as murine leukemia virus (MuLV), mouse mammary tumor virus (MMTV), and murine sarcoma virus (MSV); rat sarcoma virus (RaSV); and primate retroviruses such as human T-cell lymphotropic viruses 1 and 2 (HTLV-1, 2), and simian sarcoma virus (SSV). Many other suitable retroviruses are known to those skilled in the art. A taxonomy of retroviruses is provided by Teich, in Weiss, et al. eds., in RNA Tumor Viruses, 2nd ed., Vol. 2 Cold Spring Harbor Laboratory, New York, pp. 1-16 (1985). For example, a retroviral vector may be constructed so as to lack one or more of the replication genes such as gag (group-specific antigen), pol (polymerase) or env (envelope) protein encoding genes. The resulting recombinant retrovirus would thus be capable of integration into the chromosomal DNA of an infected host cell, but once integrated, be incapable of replication to provide infective virus, unless the cell in which it is introduced contains another proviral insert encoding functionally active trans-acting viral proteins. Methods for producing infectious but replication deficient viruses are known in the art such as described in Mann, et al. 1983 Cell 33:153, and Miller, et al. 1986 Mol Cell Biol 6:2895.

The immortalizing genes, preferably oncogenes can be any that are known in the art. The oncogenes are preferably chosen according to the synergy amongst them in cellular transformation, and their ability to transform the target cells. Further, the large sizes of some oncogenes may affect their inclusion on the same vector. In order to provide transforming capability, the RNA or DNA constructs of the present invention incorporate at least two oncogenes, which can be derived from viral, cellular genomes, mammalian or avian chromosomal RNA or DNA. Partial lists of oncogenes are provided by Bishop, et al., in: RNA Tumor Viruses Weiss, et al. eds., Vol. 1, Cold Spring Harbor Laboratory, New York, pp. 1004-1005 (1984), and Watson et al., in Molecular Biology of the Gene, 4th Ed., Vol II (Benjamin Cummings, Menlo Park, Calif., USA) p. 1037. Included are the known oncogenes such as jun, src, yes, abl, fps, fes, fms, ros, kit, mos, raf, H-ras, K-ras, sis, SV40 T-antigen (SV40 T-Ag), HPV E6, HPV E7, Adenovirus EA, Her2/neu, C-erbB2, C-erB3, myc, myb, fos, ski and erbA. Many oncogene products have tyrosine-specific protein kinase or serine/threonine protein kinase activity, or appear to be homologues of growth factors, growth factor receptors, or are nuclear proteins with unknown function. Many oncogenes can be obtained from public collections of deposited biological materials.

In a preferred embodiment the SV40 T-antigen, adenovirus EA, or human papilloma virus E6 and/or E7 oncogenes are used.

Retroviral vectors capable of expressing multiple genes under the control of a promoter in eukaryotic cells are known in the art. For example, one method utilizes the ability of ribosome to reinitiate translation by a scanning mechanism after encountering a stop codon (Kozak, M., 1989 J Biol Chem 108:229-41). This has been exploited to develop retroviral vectors in which two genes driven by the same promoter are efficiently expressed by being arrayed in close proximity (Levine, F., et al. 1991 Gene 108:167-74).

Transfection may be achieved by any of a variety of means described above and known to those of ordinary skill in the art including, but not limited to, retroviral infection. In general, plated cells may be transfected by infection with a suitable retrovirus, adenovirus, or adeno-associated virus.

### Utility of Cell Lines

The immortalized, stable cell lines of the invention are useful in the following respects.

### (1) Study of Inner Ear Fluid Homeostasis

The cell lines of the present invention are envisioned to be useful in studies of inner ear diseases (e.g., Meniere' s disease). Various agents may be screened for their ability to prevent or treat improper regulation of endolymph, as well as for their ability to induce proper fluid homeostasis. These cells can be used to investigate aspects of inner ear fluid regulation, ion channel function and ion transport as detailed in Examples 3 and 4.

### (2) Discovery of Specific Biomarkers for Disorders of the Inner Ear

Specific biomarkers may be identified for inner ear disorders (e.g., Meniere's Disease and auto-immune ear disease (AIED)). Innovative management and treatment strategies may be devised for inner ear immune-mediated inflammatory disorders of unknown etiologies where currently there are neither biomarkers nor cures.

Molecular profiling via genomics, proteomics, and metabonomics (e.g., the study of metabolic responses to drugs, environmental changes and diseases) has opened new windows to study disease states and biological systems. It has also provided exciting opportunities for novel applications in clinical research as well as in drug discovery and development. Genomic experiments generate high-dimensional data via parallel measurements about the disease state. The disease state that is traditionally described by a set of clinical parameters is now also described by its genomic transcriptional profile, which is defined as its genome-wide gene expression.

Gene expression profiling of mRNA with microarray technologies can suggest biomarkers that are then measured via other technologies. It is also possible to make mini-arrays containing a small number of biomarker genes for this purpose. However, this scenario is sensible only if the selection of these biomarker genes is already validated; otherwise genome-wide profiling remains the best choice as one can always refine the biomarker selection at a later stage when more data are accumulated.

Multiple techniques are known in the art to identify differences in mRNA expression between cell populations including but not limited to DNA microarrays, differential display, nucleic acid subtraction, serial analysis of gene expression (SAGE), and Reverse Transcriptase-Polymerase Chain Reaction (RT-PCR). Differences in protein expression between cell populations can also be determined, e.g., by antibody arrays and mass spectroscopy.

In some embodiments, gene expression profiling is used to identify disease-specific biomarkers by comparing gene expression in different ES cell lines (e.g., comparing gene expression in ES cells derived from a subject with an inner ear disease to gene expression in ES cells from a normal subject).

### (3) Studies of Cellular Biochemistry

Biochemical changes (e.g., in intracellular pH and calcium levels) are correlated with cell growth and action of exogenous agents. To study intracellular pH and calcium levels, cells in suitable culture vessels are exposed to fluorescent indicator dyes and then fluorescence emissions are detected with a fluorescence spectrophotometer, detailed by Grynkiewicz, et al. 1985 J Biol Chem 260:3440-3450, incorporated by herein in its entirety by reference thereto.

### (4) Studies of Cellular Responses to Growth Factors and Production of Growth Factors

The cells may be used to identify and purify growth factors important for growth and differentiation of human ES cells. For example, cellular growth and differentiation can be investigated when the cells are cultured using a serum-free medium with deficiency of a specific factor such as retinoic acid, hydrocortisone and triiodothronine.

### (5) Studies of Intracellular Communication

Intracellular communication can be investigated, e.g., by dye scrape loading assays, for example as detailed by El-Fouly, M. H. 1987 Experimental Cell Research 168:422-430, incorporated by herein in its entirety by reference thereto. To determine whether the cells growing in vitro have the ability to communicate via gap junctions, the cultures may be scraped, e.g., with a scalpel in the presence of a fluorescent dye in the growth medium. Cells at the edge of the wound are mechanically disrupted and therefore take up dye; whether intercellular communication has occurred may be ascertained by determining whether cells distant from the wound also contain dye.

### (6) Characterization of Cell Surface Antigens

The cells are incubated with an antibody against the cell surface antigen of interest, and then reacted with a second antibody which is conjugated to a fluorescent dye. The cells are then evaluated using a fluorescence activated cell sorter to determine whether they are fluorescent and therefore possess the cell surface antigen. For example, the cells can be tested for the presence of pendrin, aquaporins, hyaluronic acid synthases, toll-like receptors, ion channels and mucin genes; which are characteristics of endolymphatic sac epithelial cells Immunolabeling and ultrastructural studies are described in Examples 7-9.

### (7) Kits

Kits for screening agents and for any other usage as described herein are easily assembled, comprising container(s) containing the cell line(s) of the present invention, and optionally media for propagating cells, and reagents and/or apparatus for detecting morphological, physiological and/or genetic responses in the cell lines. Other components routinely found in such kits may also be included together with instructions for performing the one or more assays described above.

### Example 1

### Immortalization of Endolymphatic Sac Cells

Primary cell culture preparation of endolymphatic sac (ES) epithelial cell has been described (Linder et al, 2001 “In vitro growth of human endolymphatic sac cells: a transmission electron microscopic and immunohistochemical study in patients with vestibular schwannoma and Meniere's disease” Owl Neurotol 22(6):938-943). Briefly, human ES were excised during translabyrinthine acoustic neuroma surgery at Uppsala University Hospital with an institutional approval. The intraosseous portion of the human ES was drilled out, leaving a thin, movable eggshell layer of bone on its anterior surface. The human ES was separated from the posterior bony surface with a mucosal knife and was cut with a pair of scissors at the external aperture of the vestibular aqueduct. Thus, only the intraosseous portion of the human ES was retained. The sample was then cut into small pieces (<1 mm3) and incubated at 4° C. in 0.17% trypsin in PBS for 15-18 h. The resulting suspension of single cells and small aggregates were transferred to a test tube, and an equal volume of growth medium was added. The cells were centrifuged for 5 min at 1,000 rpm, resuspended in growth medium, and seeded into a 35-mm diameter fibronectin and collagen coated tissue culture dish. The cells were grown in a 3:1 mixture of Dulbecco's modified Eagle medium and Ham's F12 medium (Gibco BRL) supplemented with 10% fetal calf serum (FCS) (Hyclone), 0.4 μg/mL hydrocortisone (Sigma), 10 μg /mL human epidermal growth factor (EGF) (Austral Biologicals), 10⁻¹⁰ M cholera toxin (Sigma), 5 μg/mL Zn-free insulin (Lilly Research Laboratories), 24 μg/mL adenine (Sigma), and 2×10⁻⁹ M 3,3,5-triiodo-L-thyronine (Sigma).

We immortalized cultured primary cells of human ES epithelial cells using a retrovirus containing the E6/E7 genes of human papilloma virus type 16. The cells were kept in G418-containing selection media for 14 days for selection. For colony isolation, cloning rings were used to encircle the colonies; and the cells were harvested. We selected two clones that were passaged over 50 times without any evidence of a reduced proliferative capacity. Karyotypic analysis showed that the ES cell line has no major chromosomal anomaly. The stable ES cell line appeared to express markers characteristic of epithelial cells. We also found that our ES cell line expresses pendrin, aquaporins, hyaluronic acid synthases, toll-like receptors, ion channels and mucin genes; which are characteristics of endolymphatic sac epithelial cells.

### Example 2

### Immunohistochemical Characterization

To assure that the cells purified and expanded from primary ES cells are ES cells, the characteristics of the primary culture cells are stained with ES cells marker. The culture cells at every other passage are stained for ES cell specific antibody.

The cultured ES cells are plated in laminin-coated chamber slides. Forty-eight hours later, plated cells are fixed in methanol/ethanol (1:1) at −20° C. for 15 minutes. The slides are then washed three times with phosphate-buffered saline (PBS) and blocked with 10% normal goat serum (NGS) in PBS for 20 min at room temperature. Cells are incubated for one hour with primary antibody, which is a marker specific for ES cell types. For example, the cells can be labeled with rabbit antibodies against pendrin, aquaporins, hyaluronic acid synthases, toll-like receptors, mucin genes and other ion channels such as NKCCs, ENaCs, NHEs and TRPVs. After washing three times with PBS, secondary fluorescein-isothiocyanate (FITC)-conjugated goat anti-rabbit antibody is added and incubated for 30 min at room temperature. After three washes with PBS, the slides are covered with antifade/pipidien iodine or regular permount and viewed under a Zeiss fluorescent microscope (Zeiss, Germany).

### Example 3

### Ion Channel Function

Epithelial sodium channels are expressed in ES cells. To characterize epithelial sodium channel (ENaC)-dependent current, the ES cell line is grown on Snapwell permeable supports with surface areas of 1.13 cm² (Costar Co., Cambridge, Mass., USA) for 7 days. After confluence, the cells form a tight epithelium. The cells are mounted in Us sing chambers (World Precision Instruments, Sarasota, Fla., USA). The epithelium is bathed on both sides with 5 mL of warmed (37° C.) standard biocarbonate solution circulated by gas lifts with 95% O₂ and 5% CO₂. Solution pH is maintained at 7.4. The epithelial culture is voltage clamped with an automatic voltage clamp and the short-circuit current (I_{sc}) is measured. Measurement of amiloride- and adenosine triphosphate (ATP)-dependent current is performed in Ussing chambers and a 15-minute equilibration is performed to stabilize the transepithelial current. Then, to measure ENaC-dependent current, amiloride (10 μM) is added to the luminal bath. After a 5- to 10-minute interval to allow the current to stabilize, ATP (100 μM) is added to the luminal bath.

### Example 4

### Water Permeability Measurement

For water permeability measurement, an ES cell line is plated in 96-well clear bottom of black-walled microplates (Costar) at a density of 20000 cells per well in F-12 Ham's medium supplemented with 10% FBS and 0.5 mg/mL G418. After incubation at 37° C. in an atmosphere containing 5% CO₂ with 90% humidity for 18 h, the cells in 96-well plates are washed twice in PBS buffer (200 μL/wash), leaving 100 μL PBS after the last wash. Measurement is performed on a FLUOstar Optima plate reader (BMG Technology) equipped with syringe pumps and excitation and emission filters. Cells in each well of the plate are assayed individually for osmotically driven (e.g., aquaporin-mediated), water influx across the plasma membrane that dilutes the cytoplasmic Cl⁻ by recording fluorescence increase continuously (0.2 s per point) for 2 s (baseline) and then for 21 s (water transport into the cells) after rapid injection (<1 s) of 100 μL distilled water. Water permeability is expressed as half time (t_{1/2}) from water injection to the point when the cytoplasmic fluorescence reaches the maximum. A smaller t_{1/2} represents a higher water permeability.

### Example 5

### Secretion of Osmotically Active Substances

We found that our ES cell line secretes osmotically active and viscous substances. For example, viscosity of the substance derived from the cells can be measured using an Ubbelohde viscometer (VWR) after collection of the supernatant. Osmolality of the substance can be measured using a freezing point osmometer (Precision Systems, Natick, MA). To determine the factors influencing secretion of osmotically active and viscous substances, the cells can be cultured using a serum-free medium with deficiency of a specific factor such as retinoic acid, hydrocortisone and triiodothronine.

### Example 6

### Regulation of Hyaluronic Acid Synthases

We identified hyaluronic acid among the osmotically active substances derived from our ES cell line. Three isoforms of hyaluronic acid synthases (HASs) are identified in humans. For example, transcriptional regulation of each HAS can be determined using real-time quantitative RT-PCR analysis after extraction of total RNAs from the ES cell line. Protein levels of each HAS can be measured using immunobloting after extraction of total proteins from the ES cell line. To determine the factors regulating each HAS, the cells can be cultured using a serum-free medium with deficiency of a specific factor such as retinoic acid, hydrocortisone and triiodothronine.

### Example 7

### Regulation of AQP2 and AQP3 in Immortalized Human ES Epithelial Cells

The inner ear can function properly only if the chemical equilibrium and volume of inner ear fluids are maintained. The disruption of inner ear homeostasis results in many inner ear disorders such as Meniere's disease. The endolymphatic sac (ES) is believed to be involved in homeostasis of endolymph such as ion and fluid regulation. Aquaporins (AQPs) function like a molecular water valve, allowing water to move along a specific direction. AQP1, 2, 3, 4, and 6 are all found in the ES and may have an important role in endolymph fluid homeostasis. In this study, we aim to demonstrate if ES epithelial cells regulate AQP2 and AQP3 in response to vasopressin and dexamethasone, respectively. Immunolabeling showed that AQP2 and AQP3 are differentially expressed in the murine ES epithelium. RT-PCR analysis of the ES cells showed the expression of AQP2 and V2R and the vasopressin-induced AQP2 up-regulation. Luciferase assays demonstrated that dexamethasone induces AQP3 up-regulation in the ES cells. Fluorescence-quenching assays showed that calcein fluorescence is increased by hypertonic PBS, but is decreased by isotonic PBS. It was found that water permeability of ES cells is affected upon exposure to vasopressin and dexamethasone. Taken together, our findings indicate that ES epithelial cells are invovled in homeostasis of endolymph through regulation of AQP2 and AQP3. We believe that our ES cell line will provide an in vitro model enabling us to better understand the molecular pathogeneis of Meniere's disease.

### Example 8

### Characterization of the Human ES Cell Line Secreting Osmotically Active Substances

The epithelium of endolymphatic sac (ES) is formed by two distinct types of cells: ribosome-rich dark and mitochondria-rich light cells in rodents. The light cells are believed to be involved in fluid regulation whereas the dark cells, in protein synthesis and secretion. A homogeneous substance is found in the lumen of vertebral ES, which is stained with basic aniline dyes and appears to contain glycoaminoglycans with an osmotic activity. The accumulation and degradation of a homogeneous substance is believed to contribute to endolymph fluid homeostasis. Here, we describe the establishment of a human ES cell line preserving a secretory function of osmotically active substances. Human ES epithelial cells were immortalized with infection of a retrovirus containing the E6/E7 genes of human papilloma virus type 16. The immortalized ES cells appeared to preserve the characteristics of epithelial cells such as cobblestone-like appearance with anchorage-dependency and expression of ZO-1. Ultrastructural studies showed that our cell line forms a junctional complex and has microvilli-rich and -poor subpopulations. FACS analysis demonstrated that our ES cell line has subpopulations according to pendrin expression and mitochondrial richness. Viscometry showed that the immortalized ES cells secret highly viscous substances in response to the cocktail of growth factors. Osmometry showed that the viscous substances have a water-retaining activity. Altogether, our findings indicate that our ES cell line preserve the normal ES epithelial characteristics and will enable us to elucidate molecular mechanisms related to secretory regulation of osmotically active substances, which may be involved in endolymph fluid homeostasis.

### Example 9

### MUC1 Expression in Endolymphatic Sac Epithelial Cells

The endolymphatic sac (ES) is believed to be involved in homeostasis of inner ear endolymph. The disruption of inner ear homeostasis results in many inner ear disorders such as endolymphatic hydrops and Meniere's disease. A homogeneous substance found in the lumen of the vertebral ES appeared to contain glycoproteins and polysaccharides. Mucins are a family of heavily glycosylated proteins (glycoconjugate) produced by epithelial tissues in most metazoans and play a role in lubrication, chemical barriers and innate immunity against pathogen. In this study, we aimed to investigate the expression of mucin genes such as MUC1 in the ES and establish an in vitro model of human ES epithelial cells. RT-PCR analysis and immunolabeling showed the expression of Mucl in the rat ES. We immortalized the human ES epithelial cells through infection with a retrovirus containing the E6/E7 genes of human papilloma virus type 16, which appeared to preserve the characteristics of normal ES epithelial cells such as cobblestone-like appearance, anchorage-dependency, and expression of ZO-1. Ultra-structural studies showed that our human ES epithelial cell line forms a junctional complex and has microvilli-rich and -poor subpopulations. Moreover, our human ES epithelial cell line appeared to preserve the expression of MUC1 and up-regulate MUC1 in response to the cocktails of growth factors. Altogether, our findings indicate that we successfully established the in vitro model of human ES epithelial cells, which will enable us to elucidate molecular mechanisms related to endolymph fluid homeostasis. As far as we know, we first demonstrated the expression of MUC1 in the ES, but the specific fuction of MUC1 in the ES remains to be further investigated.

### Example 10

Establishment of Cell Lines from the Human Middle and Inner Ear Epithelial Cells

The middle ear infection is the most common childhood infection. In order to elucidate the cell and molecular mechanisms involved in bacterial recognition and innate immune response, we have established a stable human middle ear cell line, which has contributed to the current knowledge concerning the molecular pathogenesis of the middle ear infection. The inner ear, a sensory organ responsible for hearing and balance, is filled with inner ear fluid, and disturbance of the fluid homeostasis results in dizziness and hearing impairment. It has been suggested that the endolymphatic sac (ES) may play a critical role in the fluid homeostasis of the inner ear. We have established a stable human ES cell line and have undertaken cellular and molecular characterization of this cell line.

The middle ear infection (otitis media) is the most common pediatric infectious disease and is the most common reason for physician's office visits and antibiotic prescriptions (Vergison A (2008) Microbiology of otitis media: a moving target. Vaccine 26(Suppl 7):G5-G10). It was estimated to cost the US alone roughly five billion dollars per year for the management of otitis media (OM) in 1996 (Gates G A (1996) Cost-effectiveness considerations in otitis media treatment. Otolaryngol Head Neck Surg 114(4):525-530). However, exact molecular mechanisms involved in the pathogenesis of OM are poorly understood (Lim D J, Chun Y M, Lee H Y, Moon S K, Chang K H, Li J D, Andalibi A (2000) Cell biology of tubotympanum in relation to pathogenesis of otitis media—a review. Vaccine 19 (Suppl 1):S17-S25). In order to elucidate a mechanism involved in bacterial recognition and innate and adaptive immune response, we have established a stable human middle ear cell line, which contributed to the current knowledge concerning the cell signaling involved in the pathogenesis of the middle ear infection.

The inner ear, which contains unique sensory organs responsible for hearing and balance, is filled with lymph fluids such as perilymph and endolymph (Lim D J (1981) Middle ear and inner ear structure and biological function. In: Bernstein J M, Ogra P L (eds) Immunology of the ear. Raven, New York, pp 1-38). The perilymph is characterized by high Na+ ion, whereas the endolymph is characterized by high K+ ion (Kim S H, Marcus D C (2010) Sodium homeostasis in the inner ear. In: Kim H N, Chung M H, Lee W S (eds) Current opinion on sensorineural hearing loss. Gunsa, Seoul, pp 41-63). Disturbance of fluid homeostasis results in hearing impairment and vertigo. Particularly, Meniere's disease (MD), characterized by fluctuating hearing loss, tinnitus, and dizziness, is considered to be an example of inner ear fluid homeostasis disorders. While the underlying causes of MD are not yet known, it has been suggested that the endolymphatic sac (ES) may play a critical role in the pathogenesis of MD (Lim D J, Moon S K (2010) The endolymphatic sac structure and function and their clinical implications: a review. In: Kim H N, Chung M H, Lee W S (eds) Current opinion on sensorineural hearing loss. Gunsa, Seoul, pp 65-84; Lim D J (1999) Ultrastructure of the endolymphatic duct and sac in normal and Meniere's disease. In: Harris J P (ed) Meniere's disease. Kugler, Hague, pp 175-193). It has been suggested that the ES may play an important physiologic function to regulate endolymph fluid homeostasis and also may function as an immune organ (Tomiyama S, Harris J P (1987) The role of the endolymphatic sac in inner ear immunity. Acta Otolaryngol 103 (3-4):182-188). In order to elucidate the cell and molecular mechanisms involved in the physiology and pathophysiology of MD, it is desirable to establish a stable human ES cell line. We report the progress thus far, which we have made in the characterization of this cell line.

### Functional Morphology of the Ear

### Middle Ear Epithelium

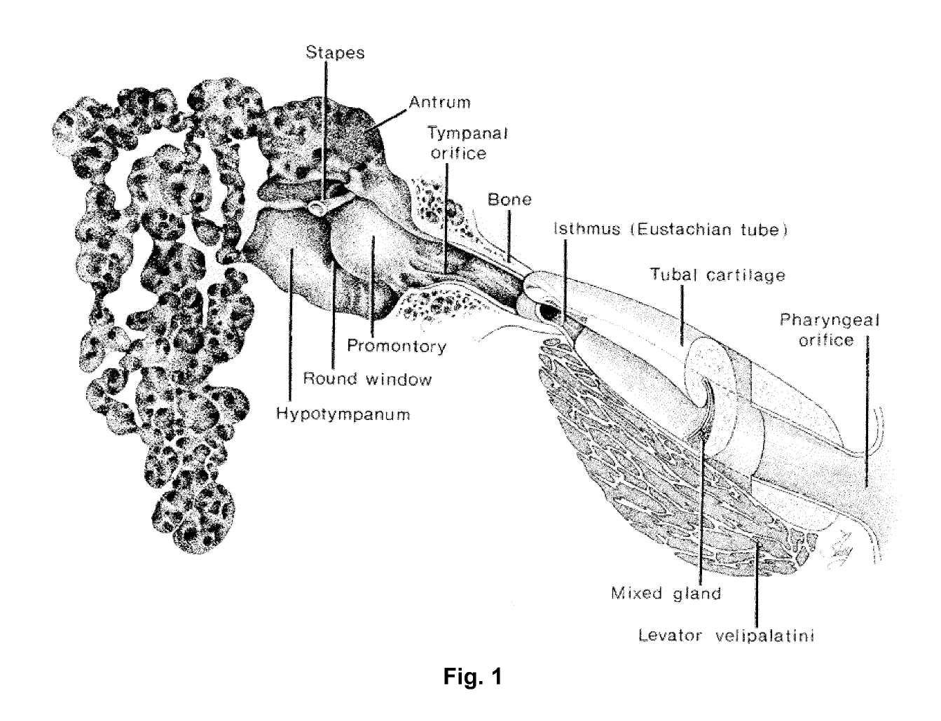

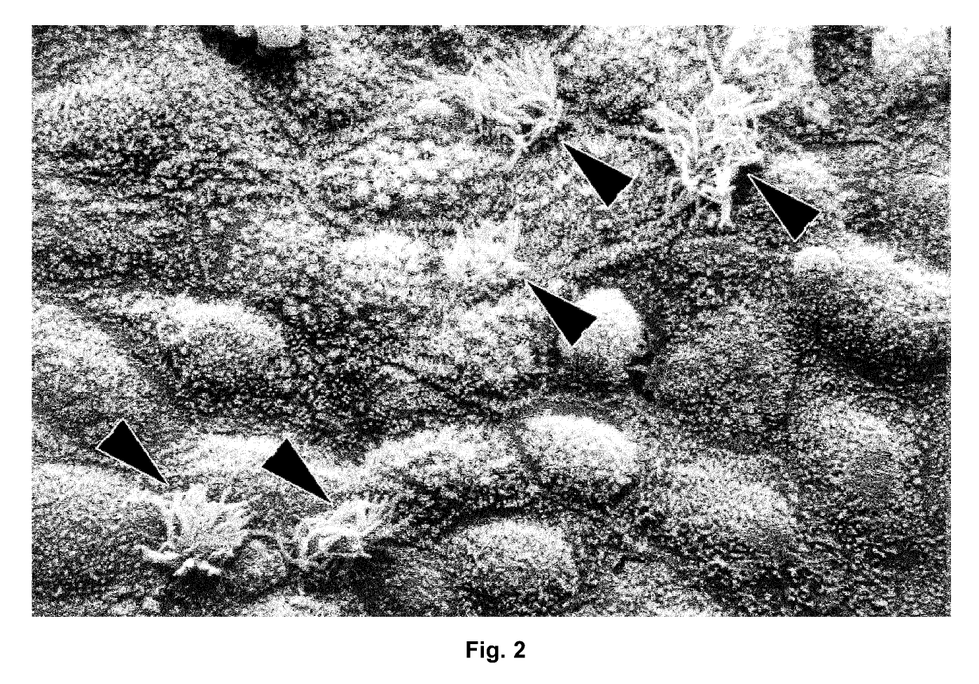

The middle ear cavity is connected to the oropharynx through eustachian tube, which provides pressure regulation and aeration of the tympanic cavity and the mastoid cavity (FIG. 1). The lining mucosa of the middle ear consists of ciliated cells, secretory cells, nonsecretory cells, and basal cells (FIG. 2) (Lim D J, Paparella M M, Kimura R S (1967) Ultrastructure of the eustachian tube and middle ear mucosa in the guinea pig. Acta Otolaryngol 63(5): 425-444; Lim D J (1974) Functional morphology of the lining membrane of the middle ear and eustachian tube: an overview. Ann Otol Rhinol Laryngol 83 (Suppl 11): 15-26). The middle ear epithelial cells vary depending on the location.

Near the eustachian tube orifice, these cells are columnar or cuboidal, and they are becoming flat epithelial cells toward the mastoid cavity. The number of secretory cells is proportional to the number of ciliated cells. In the promontory region, the epithelial cells are largely cuboidal. The eustachian tube consists of two parts: bony parts closer to the tympanic cavity and the cartilaginous part connected to the oropharyngeal opening. This latter part has rich mixed glands. Tubal cartilage is closed most of time, but opens intermittently to equate pressure and aeration. The lining epithelium is largely ciliated and secretory cells and its major function is to transport unwanted particles trapped in the mucous blanket by mucociliary transport system. The tubal dysfunction is believed to be involved with the risk factor for OM in children during the winter months when upper respiratory infections (URI) are prevalent (Lim D J, DeMaria T F, Bakaletz L O (1987) Functional morphology of the tubotympanum related to otitis media: a review. Am J Otol 8(5):385-389). URI is known to impair or destroy mucociliary epithelium, leading to the tubal dysfunction. The tubal dysfunction is believed to result in high negative pressure of the tympanic cavity. Therefore, the bolus of mucus containing pathogenic bacteria may enter into the tympanic cavity when the tube attempts to open.

The proportion of the ciliated and secretory cells reflects the history of inflammation in the middle ear, suggesting the middle ear epithelial cell type is in dynamic state (Lim D J, Birck H (1971) Ultrastructural pathology of the middle ear mucosa in serous otitis media. Ann Otol Rhinol Laryngol 80(6):838-853). The epithelial cells are also known to elaborate antimicrobial molecules, which constitute to the innate immune defense mechanism (Lim D J, Chun Y M, Lee H Y, Moon S K, Chang K H, Li J D, Andalibi A (2000) Cell biology of tubotympanum in relation to pathogenesis of otitis media—a review. Vaccine 19 (Suppl 1):S17-S25).

### Inner Ear

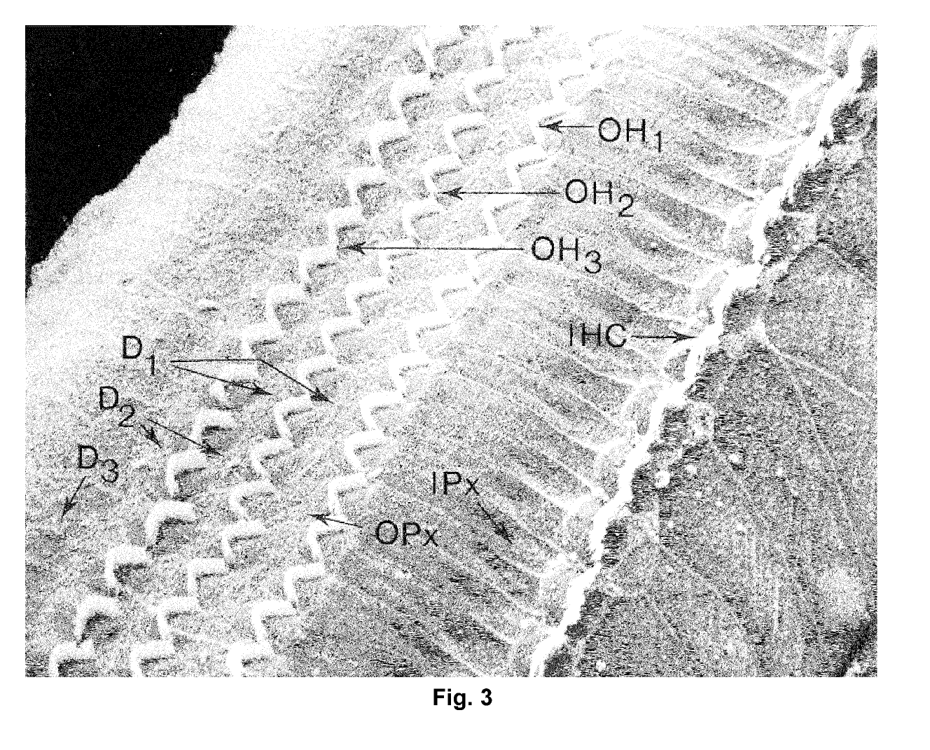

As to the functional morphology of the inner ear, the auditory sensory organ (organ of corti of the cochlea) is a unique sensory structure composed of one row of inner hair cells (IHC) and three (or four) rows of the outer hair cells in mammals (FIG. 3) (Lim D J (1980) Scanning electron microscopic morphology of the ear. In: Paparella M M, Shumrick C A (eds) Textbook of otolaryngology, vol 1. W B Saunders, Philadelphia, pp 439-469; Lim D J (1986) Functional structure of the organ of corti: a review. Hear Res 22:117-146; Lim D J, Lane W C (1969) Three-dimensional observation of the inner ear with the scanning electron microscope. Trans Am Acad Ophthalmol Otolaryngol 73(5):842-872; Lim D J, Lane W C (1969) Cochlear sensory epithelium. A scanning electron microscopic observation. Ann Otol Rhinol Laryngol 78(4):827-841). The IHC are considered the primary sensory cells responsible for transmitting electrical signals to the brain, whereas the outer hair cells are responsible for fine tuning (frequency resolution) of the hearing through motile activity of the sensory cells, by regulating their lengths. Electrical stimulation of the outer hair cells induces motor activity through their unique motor proteins (known as Prestin) embedded in the membrane structure (Matsumoto N, Kalinec F (2005) Prestin-dependent and prestin-independent motility of guinea pig outer hair cells. Hear Res 208(1-2):1-13).

In addition, the sensory organ is bathed in different fluid compartments. The endolymph compartment (scala media) is filled with K+ ion-rich fluid, whereas the perilymph compartments, composed of scala vestibuli and scala tympani, contain Na⁺ ion-rich fluid. The perilymph space is communicating with the CSF through the cochlea aqueduct.

### Endolymphatic Sac

The endolymphatic compartment is connected to the blind sac known as ES through endolymphatic duct (ED). The ES is composed of intraosseous and intradural parts. In human, the ES is composed of multiple interconnecting tubules (Linthicum F H Jr, Tian Q, Milicic M (1995) Constituents of the endolymphatic tubules as demonstrated by three-dimensional morphometry. Acta Otolaryngol 115(2):246-250), occasionally filled with dense PASpositive materials (Tian Q, Rask-Andersen H, Linthicum F H Jr (1994) Identification of substances in the endolymphatic sac. Acta Otolaryngol 114(6):632-636), which is believed to be the osmotic agent required for fluid volume regulation of the endolymph system. Experimental evidences suggest that ES epithelial cells may secrete these osmotic agents. In addition, the ion transport activities of the ES involve mainly Na⁺ and Cl⁻ions. The ES epithelial cells express ion channels and cotransporters, such as ENaC, Na⁺-K⁺-2Cl-type 2 (NKCC-2) cotransporter in addition to SLC26A4, carbonic anhydrase, and Cl⁻/HCO₃⁻ exchanger (Kim S H, Marcus D C (2010) Sodium homeostasis in the inner ear. In: Kim H N, Chung M H, Lee W S (eds) Current opinion on sensorineural hearing loss. Gunsa, Seoul, pp 41-63; Akiyama K, Miyashita T, Mori T, Inamoto R, Mori N (2008) Expression of thiazide-sensitive Na⁺-Cl⁻ cotransporter in the rat endolymphatic sac. Biochem Biophys Res Commun 371(4):649-653; Akiyama K, Miyashita T, Mori T, Mori N (2007) Expression of the Na⁺-K⁺-2Cl⁻ cotransporter in the rat endolymphatic sac. Biochem Biophys Res Commun 364(4):913-917).

There are also strong evidences that the ES is the immune organ of the inner ear, containing resident macrophages and lymphocytes (Lim D J, Moon S K (2010) The endolymphatic sac structure and function and their clinical implications: a review. In: Kim H N, Chung M H, Lee W S (eds) Current opinion on sensorineural hearing loss. Gunsa, Seoul, pp 65-84), and it is suggested to play a critical role in the immune response of the inner ear (Wackym P A, Friberg U, Linthicum F H Jr, Bagger-Sjoback D, Bui H T, Hofman F, Rask-Andersen H (1987) Human endolymphatic sac: morphologic evidence of immunologic function. Ann Otol Rhinol Laryngol 96(3 Pt 1):276-281). The luminal fluid often contains dense glycoprotein substances as well as free floating macrophages. In addition to macrophages, there are reports to indicate the presence of intraepithelial lymphocytes (Rask-Andersen H, Stahle J (1979) Lymphocytemacrophage activity in the endolymphatic sac. An ultrastructural study of the rugose endolymphatic sac in the guinea pig. ORL J Otorhinol Relat Spec 41(4): 177-192).

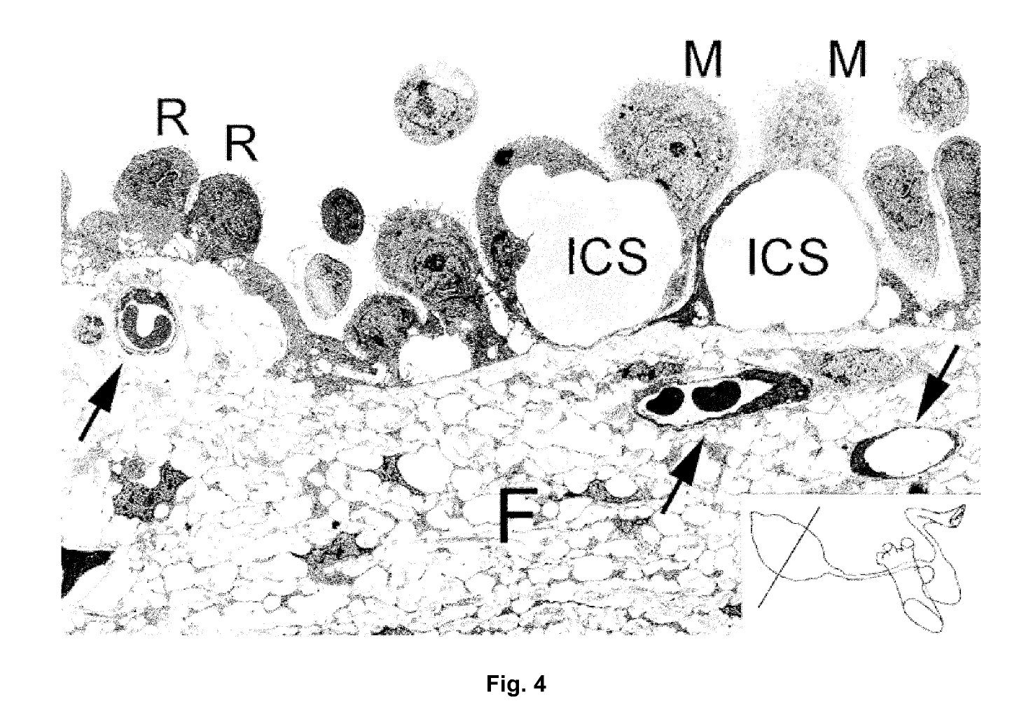

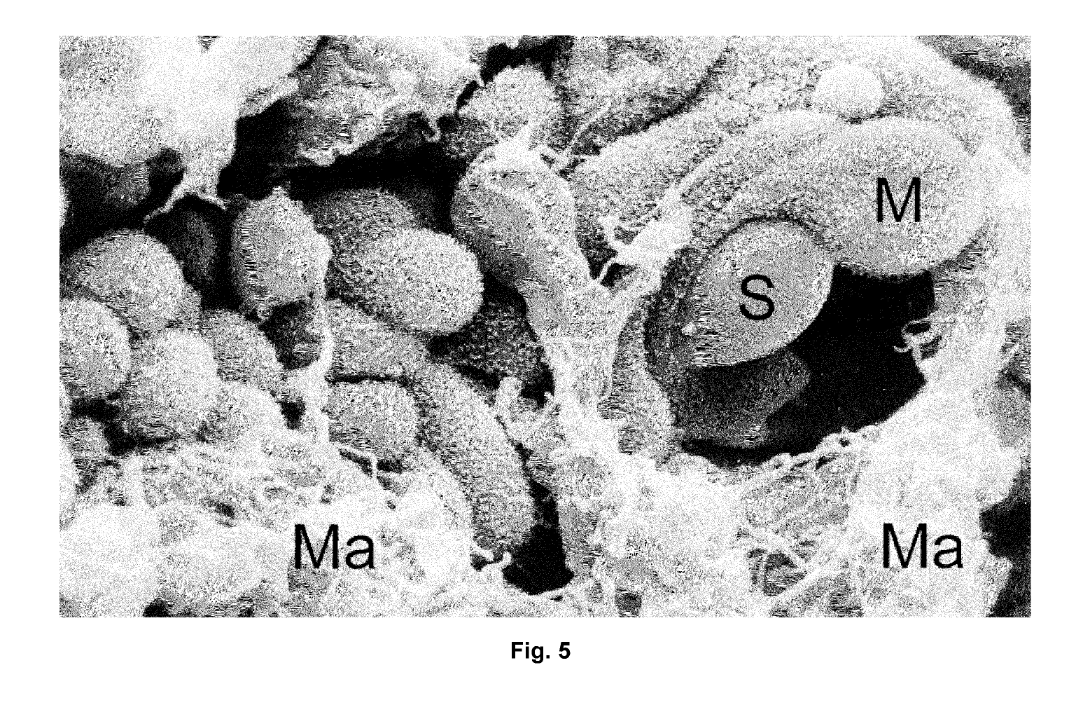

The ES consists of epithelial cells and loose subepithelial connective tissue, which is continuous with the perilympathic space of the ED. The ES epithelial cells are composed of relatively flat epithelial cells of two types (FIG. 4) (Barbara M, Rask-Andersen H, Bagger-Sjoback D (1987) Ultrastructure of the endolymphatic sac in the Mongolian gerbil. Arch Otorhinolaryngol 244(5): 284-287). Mitochondria-rich light cell is characterized by a large number of microvilli on its luminal surface, whereas mitochondria-poor ribosome-rich dark cell has a relatively smooth luminal cell surface (FIG. 5). Mitochondria-rich cell is also believed to express Pendrin and carbonic anhydrase based on the studies with immunolabeling (Peters T A, Tonnaer E L, Kuijpers W, Cremers C W, Curfs J H (2002) Differences in endolymphatic sac mitochondria-rich cells indicate specific functions. Laryngoscope 112(3):534-541). Foxi-1 gene is upstream for Pendrin expression, and Foxi-1-deficient mice develop endolymphatic hydrops and the ES of these animals lack Pendrinpositive (mitochondria-rich) cells (Hulander M, Kiernan A E, Blomqvist S R, Carlsson P, Samuelsson E J, Johansson B R, Steel K P, Enerback S (2003) Lack of pendrin expression leads to deafness and expansion of the endolymphatic compartment in inner ears of Foxil null mutant mice. Development 130(9):2013-2025).

The subepithelial connective tissue contains a large number of blood vessels and accompanying nerve supply. Sympathetic (superior cervical ganglion), parasympathetic (pterygopalatine ganglion), and somatosensory (trigeminal ganglion) innervation is known to be largely involved in regulation of the blood flow and possibly in the fluid absorption of the ES. They are either myelinated or unmyelinated (Lim DJ, Moon SK (2010) The endolymphatic sac structure and function and their clinical implications: a review. In: Kim H N, Chung M H, Lee W S (eds) Current opinion on sensorineural hearing loss. Gunsa, Seoul, pp 65-84).

### Human Middle Ear Cell Line

The middle ear epithelial cells are known to directly interact with the pathogens and pathogen-derived molecules. Since normal human middle ear epithelial cells are not easily obtainable, it is desirable to establish a stable human middle ear epithelial cell line that express important genes/gene products involved in the bacteria-host interaction and resultant inflammatory response.

### Immortalization

Small pieces of healthy human middle ear mucosa were harvested from the promontory area of the patient during translabyrinthine craniotomy. To induce proliferation of primary cells, explants of human middle ear mucosa were plated on 35-mm plastic culture dishes with a minimal volume of media, allowing them adhere to the bottom (Moon S K, Lim D J, Lee H K, Kim H N, Yoo J H (2000) Mucin gene expression in cultured human middle ear epithelial cells. Acta Otolaryngol 120(8):933-939). Cultures were maintained at 37° C. in a humidified atmosphere of 5% CO2. The culture medium used was a 1:1 mixture of bronchial epithelial growth media (BEGM; Clonetics, Walkersville, Md.) and DMEM (Gibco BRL, Gaithersburg, Md.), containing hydrocortisone (0.5 mg/mL), insulin (5 mg/mL), transferrin (10 mg/mL), epinephrine (0.5 mg/mL), triiodothyronine (6.5 mg/mL), gentamicin (50 mg/mL), and amphotericin B (50 ng/mL), all supplied by Clonetics (Walkersville, Md.), and further supplemented with EGF (25 ng/mL; Collaborative Research, Bedford, Mass.), all-trans retinoic acid (5×10 1/4 8 M; Sigma, St. Louis, Mo.), bovine serum albumin (1.5 mg/mL; Sigma), and bovine pituitary extract (1% v/v; Pel Freez, Rogers, AR).

For immortalization, we infected cells using a retrovirus containing the E6/E7 genes of human papillomavirus type 16 (Chun Y M, Moon S K, Lee H Y, Webster P, Brackmann D E, Rhim J S, Lim D J (2002) Immortalization of normal adult human middle ear epithelial cells using a retrovirus containing the E6/E7 genes of human papillomavirus type 16. Ann Otol Rhinol Laryngol 111(6):507-517). Briefly, the PAS 17 amphotropic packaging cell line, stably transfected with a replication-defective retrovirus construct (pLXSN16E6-E7), coding for HPV type 16 transforming oncoproteins E6 and E7, was grown to 70% confluence, and supernatants were collected after 24 h. Primary human middle ear epithelial cells (passage 3 at 50% confluence) were infected with 1 mL of virus stock diluted in 9 mL of fresh BEGM for 48 h. The medium was replaced with fresh BEGM and the cells were allowed to proliferate. For selection, cells were reseeded in fresh BEGM containing 0.4 mg/mL of G418 (Gibco BRL) and were kept in the selection media for 14 days. Multiple colonies were isolated using cloning rings and each clone was expanded for further characterization. One of clones appeared to be stably immortalized and was designated as HMEEC-1. The average doubling time of the HMEEC-1 was 23.8 h and appeared to preserve characteristics of epithelial cells such as expression of pan-cytokeratin, dome formation, and anchorage dependency. In addition, subcutaneous injection of HMEEC-1 cells to the nude mice did not result in tumor formation. Karyotypic analysis confirmed the immortalized cells are derived from male humans and have no major abnormality of chromosomes.

Major Research Findings Resulting from the Human Middle Ear Cell Line Secretion of Mucins

The sterility of the eustachian tube and the middle ear is maintained not only by the adaptive immune system, but also by the innate immune system such as mucociliary system and the antimicrobial molecules (Lim D J, Chun Y M, Lee H Y, Moon S K, Chang K H, Li J D, Andalibi A (2000) Cell biology of tubotympanum in relation to pathogenesis of otitis media—a review. Vaccine 19(Suppl 1):S17-S25). Mucins are high molecular weight glycoproteins that constitute the major component of mucus secretions in the eustachian tube and middle ear. The core proteins are encoded by different mucin genes (MUC genes) and we showed the expression of MUC genes such as MCU1, MUC2, MUC5AC, and MUC5B in the primary human middle ear epithelial cells (Moon S K, Yoo J H, Kim H N, Lim D J, Chung M H (2000) Effects of retinoic acid, triiodothyronine and hydrocortisone on mucin and lysozyme expression in cultured human middle ear epithelial cells. Acta Otolaryngol 120(8):944-949). Moreover, we demonstrated that HMEEC-1 cells up-regulate MUC5AC expression in response to cytoplasmic proteins of nontypeable Haemophilus influenzae via a p38 MAP kinase signaling pathway (Wang B, Lim D J, Han J, Kim Y S, Basbaum C B, Li J D (2002) Novel cytoplasmic proteins of nontypeable Haemophilus influenzae up-regulate human MUC5AC mucin transcription via a positive p38 mitogen-activated protein kinase pathway and a negative phosphoinositide 3-kinase-Akt pathway. J Biol Chem 277(2): 949-957), which is negatively regulated via the transforming growth factor beta-Smad signaling pathway (Jono H, Xu H, Kai H, Lim D J, Kim Y S, Feng X H, Li J D (2003) Transforming growth factor-beta-Smad signaling pathway negatively regulates nontypeable Haemophilus influenzae-induced MUC5AC mucin transcription via mitogen-activated protein kinase (MAPK) phosphatase-1-dependent inhibition of p38 MAPK. J Biol Chem 278(30):27811-27819). In addition, we showed that HMEEC-1 cells up-regulate MUC2 expression through the cooperation of transforming growth factor-beta-Smad signaling pathway and NF-κB activation (Jono H, Shuto T, Xu H, Kai H, Lim D J, Gum J R Jr, Kim Y S, Yamaoka S, Feng X H, Li J D (2002) Transforming growth factor-beta-Smad signaling pathway cooperates with NF-kappa B to mediate nontypeable Haemophilus influenzae-induced MUC2 mucin transcription. J Biol Chem 277(47): 45547-45557).

### Induction of Antimicrobial Agents

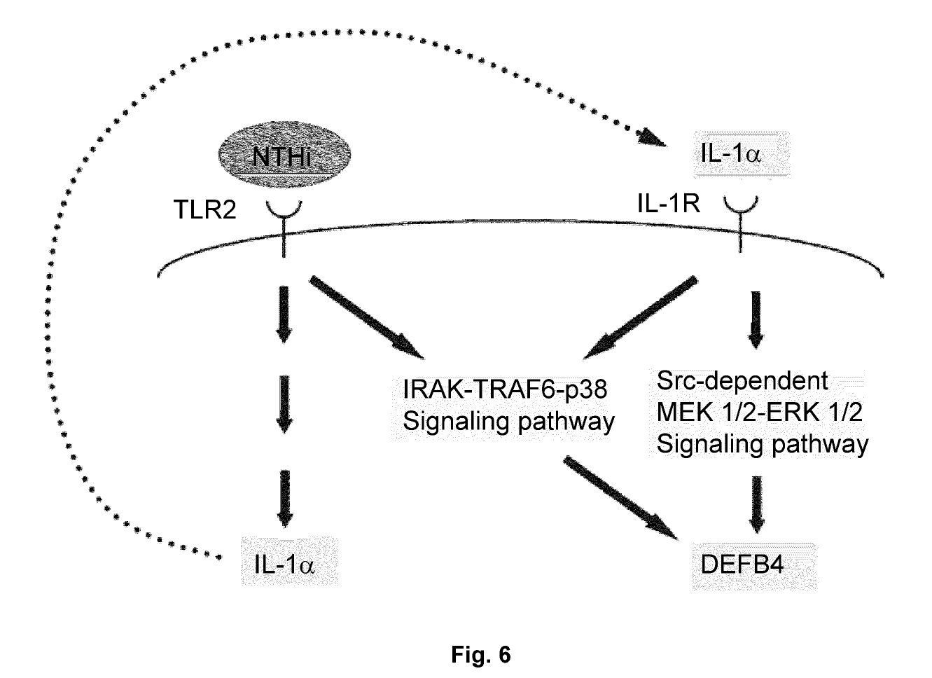

To defend against invading pathogens, the tubotympanumis known to secrete antimicrobial molecules including lysozyme, lactoferrin, and beta defensins (Lim D J, Chun Y M, Lee H Y, Moon S K, Chang K H, Li J D, Andalibi A (2000) Cell biology of tubotympanum in relation to pathogenesis of otitis media—a review. Vaccine 19(Suppl 1):S 17-S25). The beta defensins are mainly produced by epithelial cells of the skin, kidneys, and respiratory lining of nearly all vertebrates (Ganz T, Selsted M E, Lehrer R I (1990) Defensins. Eur J Haematol 44(1):1-8). The beta defensins are released upon microbial invasion and are located at the host-environment interfaces, such as mucosal surfaces and skin. Beta defensin 2, which is released from the epithelial cells in response to microorganisms or cytokines, exhibits potent antimicrobial activity against gram-negative bacteria and candida. HMEEC-1 cells were found to up-regulate beta defensin 2 in response to interleukin 1 alpha (IL-1α) via a Srcdependent Raf-MEK1/2-ERK signaling pathway (Moon S K, Lee H Y, Li J D, Nagura M, Kang S H, Chun Y M, Linthicum F H, Ganz T, Andalibi A, Lim D J (2002) Activation of a Src-dependent Raf-MEK1/2-ERK signaling pathway is required for IL-1 alpha induced upregulation of beta-defensin 2 in human middle ear epithelial cells. Biochim Biophys Acta 1590(1-3):41-51). We also found that nontypeable H. influenzae-induced up-regulation of beta defensin 2 requires a TLR2/MyD88-dependent p38 MAP kinase pathway (Lee H Y, Takeshita T, Shimada J, Akopyan A, Woo J I, Pan H, Moon S K, Andalibi A, Park R K, Kang S H, Kang S S, Gellibolian R, Lim D J (2008) Induction of beta defensin 2 by NTHi requires TLR2 mediated MyD88 and IRAK-TRAF6-p38MAPK signaling pathway in human middle ear epithelial cells. BMC Infect Dis 8:87). As shown in FIG. 6, we demonstrated that IL-1α is secreted by middle ear epithelial cells upon exposure and that it can synergistically act with nontypeable H. influenzae molecules to up-regulate beta defensin 2 via the p38 MAP kinase pathway (Moon S K, Lee H Y, Pan H, Takeshita T, Park R, Cha K, Andalibi A, Lim D J (2006) Synergistic effect of interleukin 1 alpha on nontypeable Haemophilus influenzae-induced up-regulation of human beta-defensin 2 in middle ear epithelial cells. BMC Infect Dis 6:12).

### Expression of Toll-Like Receptors

Toll-like receptors (TLRs) have been suggested to play a critical role in the recognition of various bacterial components such as lipoprotein, peptidoglycan, lipoteichoic acid, and lipopolysaccharide (Medzhitov R, Janeway C Jr (2000) Innate immune recognition: mechanisms and pathways. Immunol Rev 173:89-97). We found that HMEEC-1 cells regulate TLR2 expression and that glucocorticoids synergistically enhance nontypeable H. influenzae-induced TLR2 expression via a negative cross-talk with p38 MAP kinase (Shuto T, Imasato A, Jono H, Sakai A, Xu H, Watanabe T, Rixter D D, Kai H, Andalibi A, Linthicum F, Guan Y L, Han J, Cato A C, Lim D J, Akira S, Li J D (2002) Glucocorticoids synergistically enhance nontypeable Haemophilus influenzae-induced Toll-like receptor 2 expression via a negative cross-talk with p38 MAP kinase. J Biol Chem 277(19):17263-17270). Moreover, we demonstrated that TLR2 is involved in nontypeable H. influenzae-induced NF-kB activation through the TAK1-dependent NIK-IKK-IKB and MKK3/6-p38 MAP kinase signaling pathways (Shuto T, Xu H, Wang B, Han J, Kai H, Gu X X, Murphy T F, Lim D J, Li J D (2001) Activation of NF-kappa B by nontypeable Hemophilus influenzae is mediated by toll-like receptor 2-TAK1-dependent NIK-IKK alpha /beta-I kappa B alpha and MKK3/6-p38 MAP kinase signaling pathways in epithelial cells. Proc Natl Acad Sci U S A 98(15):8774-8779). In addition, we found the involvement of TLR2 in the recognition of nontypeable H. influenzae molecules, resulting in induction of beta defensin 2 (Lee H Y, Takeshita T, Shimada J, Akopyan A, Woo J I, Pan H, Moon S K, Andalibi A, Park R K, Kang S H, Kang S S, Gellibolian R, Lim D J (2008) Induction of beta defensin 2 by NTHi requires TLR2 mediated MyD88 and IRAK-TRAF6-p38MAPK signaling pathway in human middle ear epithelial cells. BMC Infect Dis 8:87).

### Human Endolymphatic Sac Cell Line

Because of the location of the ES deep inside the petrosal bone and the difficulty of obtaining epithelial cells, experiments using the human ES epithelial cells have been difficult to perform. Recently, we have successfully developed a human ES cell line preserving the characteristics of normal ES epithelial cells such as the expression of Pendrin.

### Immortalization

Primary cell culture preparation of ES epithelial cell has been described (Linder B, Bostrom M, Gerdin B, Rask-Andersen H (2001) In vitro growth of human endolymphatic sac cells: a transmission electron microscopic and immunohistochemical study in patients with vestibular schwannoma and Meniere's disease. Otol Neurotol 22(6):938-943). Briefly, human ES were excised during translabyrinthine acoustic neuroma surgery at Uppsala University Hospital with an institutional approval. The intraosseous portion of the human ES was drilled out, leaving a thin, movable eggshell layer of bone on its anterior surface. The human ES was separated from the posterior bony surface with a mucosal knife and was cut with a pair of scissors at the external aperture of the vestibular aqueduct. Thus, only the intraosseous portion of the human ES was retained. The sample was then cut into small pieces (<1 mm3) and incubated at 4° C. in 0.17% trypsin in PBS for 15-18 h. The resulting suspension of single cells and small aggregates were transferred to a test tube, and an equal volume of growth medium was added. The cells were centrifuged for 5 min at 1,000 rpm, resuspended in growth medium, and seeded into a 35-mm diameter fibronectin and collagen coated tissue culture dish. The cells were grown in a 3:1 mixture of Dulbecco's modified Eagle medium and Ham's F12 medium (Gibco BRL) supplemented with 10% fetal calf serum (FCS) (Hyclone), 0.4 mg/mL hydrocortisone (Sigma), 10 mg/mL human epidermal growth factor (EGF) (Austral Biologicals), 10⁻¹⁰ M cholera toxin (Sigma), 5 mg/mL Zn-free insulin (Lilly Research Laboratories), 24 mg/mL adenine (Sigma), and 2×10⁻⁹ M 3,3,5-triiodo-lthyronine (Sigma).

For immortalization, primary ES epithelial cells were infected using a retrovirus containing the E6/E7 genes of human papillomavirus type 16 as described (Chun Y M, Moon S K, Lee H Y, Webster P, Brackmann D E, Rhim J S, Lim D J (2002) Immortalization of normal adult human middle ear epithelial cells using a retrovirus containing the E6/E7 genes of human papillomavirus type 16. Ann Otol Rhinol Laryngol 111(6):507-517). Infected cells were selected using the G418-containing medium and multiple colonies were isolated. Each clone was expanded for the further characterization and one clone showing stable immortalization was designated as HESC-1. HESC-1 cells appeared to preserve the characteristics of epithelial cells such as expression of ZO-1 and cytokeratin. Karyotypic analysis demonstrated that the immortalized cells have no major abnormality in chromosomes. Although HESC-1 cells were originated from a single cell, they appeared to differentiate into two distinct subtypes, resembling ribosome-rich dark cells and mitochondria-rich light cells of the ES epithelial cells. Immunolabeling and FACS analysis showed that there are two subpopulations according to the expression of Pendrin and richness of mitochondria.

Research Findings Resulting from the Human Endolymphatic Sac Cell Line

Secretory capacity of the ES has been postulated by some authors (Adlington P (1967) The ultrastructure and the functions of the saccus endolymphaticus and its decompression in Meniere's disease. J Laryngol Otol 81(7): 759-776; Adlington P (1984) The saccus endolymphaticus in the rabbit. Further studies. J Laryngol Otol 98(9): 857-877; and Seymour J C (1954) Observations on the circulation in the cochlea. J Laryngol Otol 68(10):689-711). In about 30% of the cross-sections of the mouse ES, a small amount of homogeneous substance was observed. This homogeneous substance is believed to contain acidic protein-bound carbohydrates (Porubsky E S, Marovitz W F, Arenberg I K (1972) Presence of acidic protein-bound carbohydrates in the endolymphatic sac and duct of fetal, neonatal and adult rats, and adult humans. Ann Otol Rhinol Laryngol 81(1):76-81), proteoglycan (Friberg U, Wackym P A, Bagger-Sjoback D, Rask-Andersen H (1986) Effect of labyrinthectomy on the endolymphatic sac. A histological, ultrastructural and computer-aided morphometric investigation in the mouse. Acta Otolaryngol 101(3-4):172-182), sulfur compounds (Barbara M, Takumida M, Nilsson J, Bagger-Sjoback D, Rask-Andersen H (1989) Turnover of sulphur compounds in the endolymphatic), hyaluronan (Friberg U, Erwall C, Bagger-Sjoback D, Rask-Andersen H (1989) Hyaluronan content in human inner ear fluids. Acta Otolaryngol 108(1-2):62-67), and soluble megalin (Ishida T, Hatae T, Nishi N, Araki N (2006) Soluble megalin is accumulated in the lumen of the rat endolymphatic sac. Cell Struct Funct 31(2):77-85). Although the function of this homogeneous substance is unclear, it is suggested to be involved in the attraction of water and small water soluble cations, which could influence the regulation of fluid volume of the inner ear endolymphatic compartment (Friberg U, Wackym P A, Bagger-Sjoback D, Rask-Andersen H (1986) Effect of labyrinthectomy on the endolymphatic sac. A histological, ultrastructural and computer-aided morphometric investigation in the mouse. Acta Otolaryngol 101(3-4):172-182).

Unexpectedly, we found that our HESC-1 cells secrete viscous substance in response to EGF. Dot blot analysis showed that the supernatant of HESC-1 cells contains mucin core proteins and hyaluronan. qRT-PCR analysis demonstrated that HESC-1 cells up-regulate mucin core proteins and hyaluronic acid synthases in response to EGF. Regulatory mechanisms involved in secretion and biological functions of these molecules can be determined. The HESC-1 cells provides a basis for an in vitro model for the studies of secretory function of the ES.

### Conclusions

We have successfully immortalized epithelial cells from human middle ear and ES. The human middle epithelial cells have been used for elucidating cell signaling pathways involved in inflammatory responses including induction of beta defensins and mucin genes. Characterization of the human ES cell line has revealed the expression of genes involved in water and ion transportation, such as Pendrin and aquaporins. Particularly, the human ES cell line was found to secrete osmotically active substances.

It is understood that the examples and embodiments described herein are for illustrative purposes only and that various modifications or changes in light thereof will be suggested to persons skilled in the art and are to be included within the spirit and purview of this application and scope of any appended claims. All figures, tables, and appendices, as well as publications, patents, and patent applications, cited herein are hereby incorporated by reference in their entirety for all purposes.

What is claimed is:

1. A method for producing an immortalized human endolymphatic sac (ES) epithelial cell line, comprising:
a) providing a primary cell culture of human ES cells;
b) introducing a polynucleotide comprising an exogenous immortalizing gene into said cells;
c) selecting for immortalized cells that express the exogenous immortalizing gene and retain phenotypic properties of ES cells.

2. The method of claim 1, wherein the polynucleotide is a subgenomic fragment of a virus, selected from the group consisting of SV40, adenovirus, and human papilloma virus.

3. The method of claim 2, wherein the polynucleotide is a subgenomic fragment of a human papilloma virus, comprising the E6 and E7 genes of said human papilloma virus.

4. The method of claim 3, wherein the human papilloma virus is selected from the group consisting of types 16, 18, 31, 33, and 35.

5. The method of claim 4, wherein the human papilloma virus is type 16.

6. The method of claim 1, wherein the polynucleotide further comprises a viral or plasmid vector.

7. The method of claim 6, wherein the viral vector is selected from the group consisting of retrovirus, adenovirus, and adeno-associated virus vectors.

8. The method of claim 7, wherein the retrovirus vector comprises a replication-defective retrovirus construct.

9. A substantially pure cell line of immortalized human ES cells, which expresses an exogenous immortalizing gene.

10. The cell line of claim 9, wherein the exogenous immortalizing gene is selected from the group consisting of SV40 T antigen, adenovirus EA, and human papilloma virus E6 and E7 genes.

11. The cell line of claim 10, wherein the human papilloma virus is selected from the group consisting of types 16, 18, 31, 33, and 35.

12. A substantially pure cell line of immortalized human ES cells actively expressing the E6 and E7 gene of human papilloma virus 16, wherein the immortalized cell line maintains phenotypic characteristics of human ES cells.

13. The cell line of claim 12, which was deposited in accordance with the Budapest Treaty at the ATCC, under deposition number ______ on ______.

14. A method for determining an effect of a pharmacological agent on human ES cells, said method comprising:
a) contacting a substantially pure cell line of immortalized human ES cells which expresses an exogenous immortalizing gene, with said pharmacological agent; and
b) determining the effect of said pharmacological agent on said cell line.

15. The method of claim 14, wherein the effect is a change in cell growth.

16. The method of claim 14, wherein the effect is a change in a phenotypic characteristic of the cell line.

17. The method of claim 16, wherein the change is an increase or decrease in expression of a cellular gene.

18. The method of claim 17, wherein the cellular gene expresses a gene product selected from the group consisting of: cell cycle proteins, transcription factors, signaling molecules, cytokines, growth factors, and growth factor receptors.

19. The method of claim 14, wherein the pharmacological agent is selected from the group consisting of chemicals, drugs, hormones, cytokines, and growth factors.

20. The method of claim 14, wherein said effect is on ion channel function or ion transport.

21. A kit for screening a pharmacological agent on ES cells, comprising a substantially pure cell line of immortalized human ES cells, which express an exogenous immortalizing gene, with instructions for use.

22. A method of identifying a biomarker related to an inner ear disease comprising comparing gene expression in ES cells from a subject with an inner ear disease with gene expression in ES cells from a normal subject, wherein said ES cells from said subject with an inner ear disease and/or said ES cells from said normal subject are a cell line according to claim 9.
